(19) United States
(12) Patent Application Publication
(10) Pub. No.: US 2017/0055945 A1
(43) Pub. Date: Mar. 2, 2017

(54) ULTRASONIC PROBE AND ULTRASONIC IMAGE APPARATUS

(71) Applicant: SEIKO EPSON CORPORATION, Tokyo (JP)

(72) Inventor: Jiro TSURUNO, Okaya-shi (JP)

(73) Assignee: SEIKO EPSON CORPORATION, Tokyo (JP)

(21) Appl. No.: 15/241,569

(22) Filed: Aug. 19, 2016

(30) Foreign Application Priority Data
Aug. 24, 2015 (JP) 2015-164510

(51) Int. Cl. A61B 8/00; A61B 8/08

(52) U.S. Cl. CPC A61B 8/4444; A61B 8/461; A61B 8/52; A61B 8/4209

(57) ABSTRACT

An ultrasonic probe includes an ultrasonic device performing transmission and reception of ultrasonic waves, and an accommodation case to which an acoustic lens of the ultrasonic device is exposed and in which the ultrasonic device is accommodated. The accommodation case is formed of at least a first accommodation member and a second accommodation member, each of the first accommodation member and the second accommodation member including a suction path which is communicable with an external suction source. The first accommodation member includes, as the suction path, a first groove extending in a first direction, and the second accommodation member includes, as the suction path, a second groove extending in a second direction. The first and the second accommodation members are disposed to overlap each other, and the first direction and the second direction intersect each other in a thickness direction of the first accommodation member or the second accommodation member.

This application claims the benefit of Japanese Patent Application No. 2015-164510, filed on Aug. 24, 2015. The content of the aforementioned application is incorporated herein by reference in its entirety.

### BACKGROUND

1. Technical Field

The present invention relates to an ultrasonic probe and an ultrasonic image apparatus including the ultrasonic probe.

2. Related Art

In the related art, an ultrasonic probe is used for guiding puncturing for a living body. As an ultrasonic probe, in addition to the type of an ultrasonic probe held with the entire hand of an operator in the related art, there is a thin type ultrasonic probe formed of a thin ultrasonic vibrator.

In the ultrasonic probe, a probe comes into close contact with a skin surface so as to minimize generation of an air layer between the skin surface and the probe, and thus reduces loss of transmitted and received ultrasonic waves. Generally, as a material for minimizing the air layer between the skin surface and the probe, for example, gel for ultrasonic waves is used.

In a case where puncturing is performed by using an ultrasonic probe, an operator performs puncturing while checking a feeling from a needle tip in an unnatural attitude in which the operator holds the ultrasonic probe with one hand and inserts a puncture needle with the other hand while viewing a monitor.

JP-A-2011-172736 discloses a sticking device including an accommodator which accommodates an ultrasonic probe, a sucking section which brings the accommodator into pressing contact with a subject by sucking the subject with a suction port, a suction machine which causes suction pressure in the suction port, and an air pressure supply tube which is connected to the suction machine and the suction port and transmits suction pressure to the suction port. In JP-A-2011-172736, the sticking device stably holds the ultrasonic probe at a desired position even on a pulsating diagnosis target object.

However, in the sticking device disclosed in JP-A-2011-172736, in a case where the ultrasonic probe is brought into close contact with the subject via gel, for example, if the probe is brought into close contact by sucking air, there may be a problem that the suction port is filled with the gel, and thus close contact with the subject cannot be maintained.

Therefore, there is need for an ultrasonic probe which can be brought into close contact with skin so as to be held thereon even if gel is interposed therebetween, and an ultrasonic image apparatus including the ultrasonic probe.

### SUMMARY

An advantage of some aspects of the invention is to solve at least a part of the problems described above, and the invention can be implemented as the following aspects or application examples.

### Application Example 1

An ultrasonic probe according to this application example includes an ultrasonic device that performs transmission and reception of ultrasonic waves; and an accommodation case to which a part of the ultrasonic device is exposed and in which the ultrasonic device is accommodated, in which the accommodation case is formed of at least a first accommodation member and a second accommodation member, in which each of the first accommodation member and the second accommodation member includes a suction path which is communicable with an external suction source, in which the first accommodation member includes, as the suction path, a first groove extending in a first direction, in which the second accommodation member includes, as the suction path, a second groove extending in a second direction, and in which the first accommodation member and the second accommodation member are disposed to overlap each other, and the first direction and the second direction intersect each other in a thickness direction of the first accommodation member or the second accommodation member.

According to the ultrasonic probe, in a case where the first accommodation member and the second accommodation member are disposed to overlap each other, the first direction and the second direction intersect each other in a thickness direction, and thus a new channel as the suction path is formed. In a case where the ultrasonic probe is brought into contact with a skin surface or the like of the subject, and sucks air in a gap between the ultrasonic probe and the skin surface, the gel sucked along with air can be stored in the first groove or the second groove, for example, in a case where the gel or the like is applied on the skin surface. The sucked air is sucked by an external suction source via the new channel. With this configuration, the ultrasonic probe can be brought into close contact with the skin surface so as to be held at a desired position.

### Application Example 2

In the ultrasonic probe according to the application example, it is preferable that a plurality of the first grooves and/or the second grooves are formed.

According to the ultrasonic probe with this configuration, a plurality of the first groove and/or the second groove are formed and thus it is possible to increase a capacity for storing gel. Consequently, the ultrasonic probe can be brought into close contact with a subject so as to be held more stably.

### Application Example 3

In the ultrasonic probe according to the application example, it is preferable that a sectional shape of the first groove and/or the second groove is a triangular shape.

According to the ultrasonic probe with this configuration, even if the first accommodation member or the second accommodation member forming the accommodation case is thin, since the thickness in the sectional direction can be secured, and the rigidity of the accommodation case can be secured, it is possible to achieve thinning of the accommodation case.

### Application Example 4

In the ultrasonic probe according to the application example, it is preferable that the first accommodation member and the second accommodation member are disposed to overlap each other, and thus the first direction and the second direction are positioned in crossing relation to each other in the thickness direction.

According to the ultrasonic probe with this configuration, since the first direction and the second direction are positioned in crossing relation to and overlapped with each other, the grooves can be efficiently disposed, and thus it is possible to increase a capacity for storing gel and also to secure the rigidity of the accommodation case through overlapping. With this configuration, it is possible to simultaneously achieve conflicting objects such as an increase of the storage capacity and securing of the rigidity with good balance and at a high level.

### Application Example 5

In the ultrasonic probe according to the application example, it is preferable that the first accommodation member includes an accommodation portion that is formed on a first surface portion and in which the ultrasonic device is accommodated; a suction groove that is formed on the first surface portion along an outer circumference of the accommodation portion; the first groove that is formed on a second surface portion located on an opposite side to the first surface portion, and forms a channel; and a first communication portion that communicates with the suction groove. The second accommodation member includes the second groove that is formed on a third surface portion overlapping the second surface portion of the first accommodation member, and forms the channel by intersecting the first groove in the thickness direction; and a second communication portion that communicates with the second groove, the first communication portion communicates with the first groove, or penetrates to the second surface portion so as to communicate with the second groove, and the second communication portion penetrates to a surface portion in contact with the third surface portion, or penetrates to a fourth surface portion located on an opposite side to the third surface portion.

According to the ultrasonic probe with this configuration, in a case where the ultrasonic probe is brought into contact with a skin surface or the like of a subject, and air is sucked from the second communication portion, air in a gap between the ultrasonic probe and the skin surface is sucked via the channel formed by intersection between the second groove and the first groove, the first communication portion, and the suction groove. In a case where the ultrasonic probe is brought into contact with the skin surface of the subject via gel, the gel sucked along with air reaches the first groove and the second groove via the suction groove and the first communication portion. The gel reaching the first groove and the second groove is stored in the first groove and the second groove intersecting each other in the thickness direction, and thus the air can be sucked via the second communication portion. With this configuration, it is possible to easily form a suction path, and thus to hold the ultrasonic probe on a skin surface of a subject.

### Application Example 6

In the ultrasonic probe according to the application example, it is preferable that a plurality of the first communication portions are formed.

According to the ultrasonic probe with this configuration, a plurality of first communication portions are formed, and thus it is possible to reduce the time needed to bring the ultrasonic probe into close contact with a subject. Since the first communication portions are disposed, and thus suction pressure (negative pressure) can be substantially uniformly applied to a skin surface, it is possible to prevent the ultrasonic probe from being deviated relative to a predetermined position due to a pressure difference during suction.

### Application Example 7

An ultrasonic image apparatus according to this application example includes any one of the ultrasonic probes; a suction device that performs generation and control of suction pressure; a suction tube that connects the second communication portion to the suction device, and applies the suction pressure to the second communication portion; a processing device that controls the ultrasonic probe, and processes an input signal from the ultrasonic probe; a cable that connects the ultrasonic probe to the processing device, and performs transmission and reception of signals; and a display device that displays an image generated through processing in the processing device.

According to the ultrasonic image apparatus, since the ultrasonic probe and the suction device (suction source) are connected to each other via the suction tube, and thus suction pressure generated by the suction device is applied to the second communication portion of the ultrasonic probe, the ultrasonic probe can be brought into close contact with a subject so as to be held thereon. An input signal from the ultrasonic probe is processed by the processing device, and a generated image is displayed on the display device. Consequently, it is not necessary for an operator to hold the ultrasonic probe by hand, and thus it is possible to perform an optimal puncturing operation using both hands while checking an image on the display device in an appropriate attitude.

### BRIEF DESCRIPTION OF THE DRAWINGS

The invention will be described with reference to the accompanying drawings, wherein like numbers reference like elements.

FIG. 1 is a schematic perspective view of an ultrasonic image apparatus according to a first embodiment.

FIG. 2 is a schematic perspective view of an ultrasonic probe.

FIG. 3 is a schematic perspective view of the ultrasonic probe.

FIG. 4 is a sectional view of the ultrasonic probe taken in an A direction.

FIG. 5 is a sectional view of the ultrasonic probe taken in a B direction.

FIG. 6 is a sectional perspective view of a second accommodation member taken in the B direction.

FIG. 7 is a sectional perspective view of a first accommodation member taken in the A direction.

FIG. 8 is a sectional view of an ultrasonic probe according to a second embodiment.

FIG. 9 is a sectional view of the ultrasonic probe.

FIG. 10 is a sectional perspective view of the ultrasonic probe.

### DESCRIPTION OF EXEMPLARY EMBODIMENTS

In the present embodiment, a description will be made of an ultrasonic probe 1 and an ultrasonic image apparatus 100 including the ultrasonic probe 1 with reference to the drawings. The respective members in each drawing are illustrated in different scales so as to have recognizable sizes throughout all the drawings.

### First Embodiment

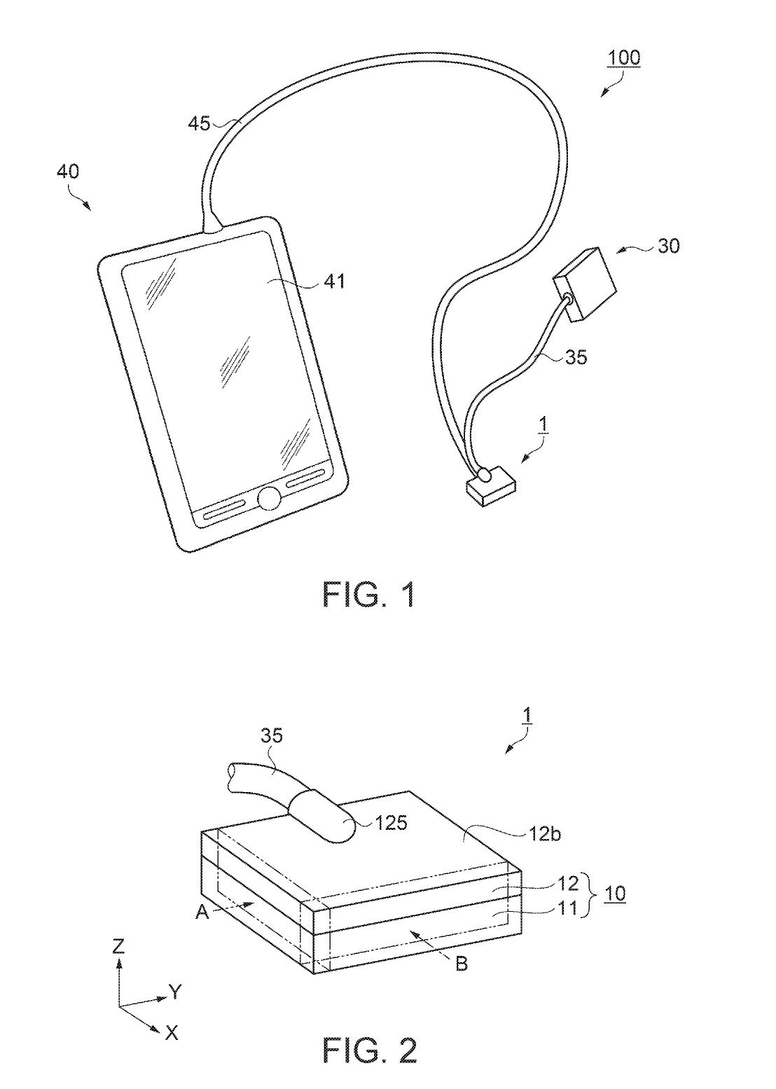

FIG. 1 is a schematic perspective view of the ultrasonic image apparatus 100 according to a first embodiment. With reference to FIG. 1, a configuration of the ultrasonic image apparatus 100 will be described.

The ultrasonic image apparatus 100 of the present embodiment is an apparatus which brings the ultrasonic probe 1 into close contact with a subject (skin surface) and holds the ultrasonic probe 1 thereon, transmits ultrasonic waves from the ultrasonic probe 1, receives reflected waves (ultrasonic waves) reflected from the inside of the subject, and analyzes data regarding the received ultrasonic waves so as to display the data as an image. An operator performs a puncturing operation with both hands while viewing the image.

The ultrasonic image apparatus 100 includes the ultrasonic probe 1, a processing device 40, and a suction device 30. The ultrasonic probe 1 and the processing device 40 are connected to each other via a flexible cable 45. The ultrasonic probe 1 and the suction device 30 are connected to each other via a flexible suction tube 35.

The processing device 40 and the ultrasonic probe 1 transmit and receive electric signals therebetween via the cable 45. The processing device 40 is provided with a display device 41, and the display device 41 displays an image (an image based on the ultrasonic waves detected by the ultrasonic probe 1) generated through processing in the processing device 40.

In a case where the ultrasonic probe 1 comes into contact with the skin surface, the ultrasonic probe 1 and the suction device 30 suck air between the ultrasonic probe 1 and the skin surface via the suction tube 35. The suction device as a suction source includes a suction machine (not illustrated) therein. The suction device 30 generates suction pressure so as to suck air between the ultrasonic probe 1 and the skin surface via a suction path in the ultrasonic probe 1 communicating with the suction tube 35. Consequently, the ultrasonic probe 1 is brought into close contact with the skin surface and is held thereon.

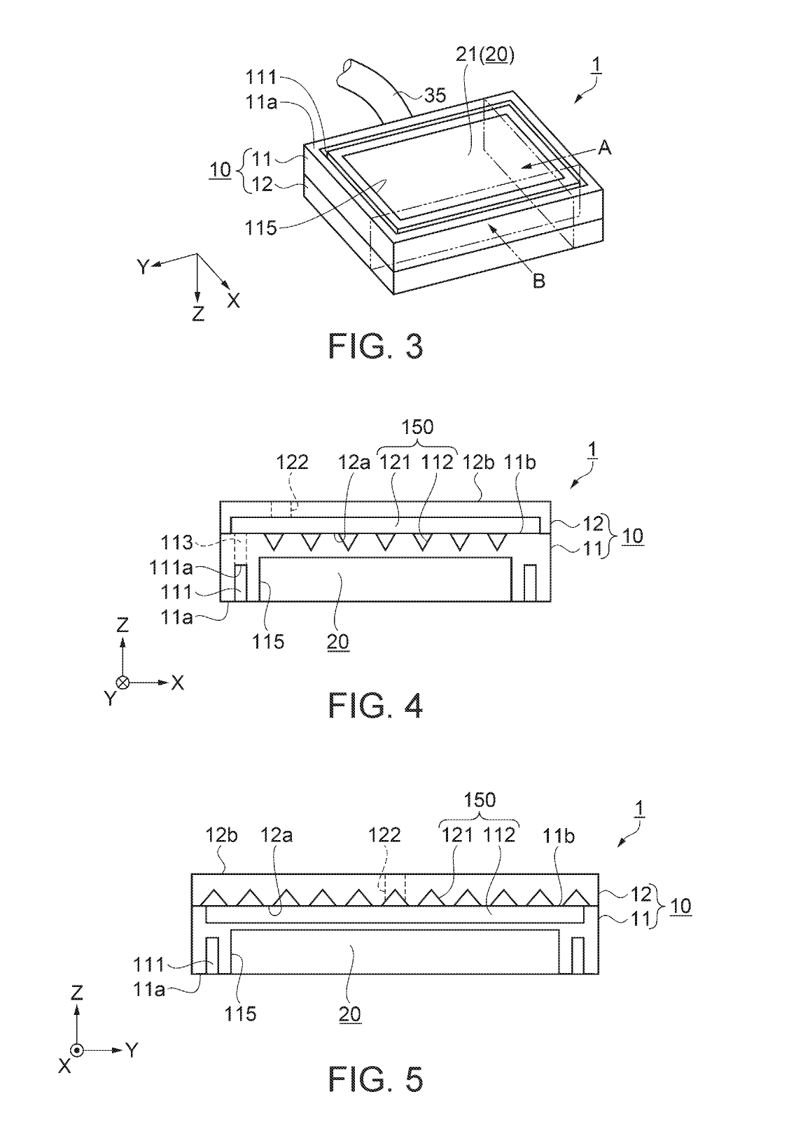

FIGS. 2 and 3 are schematic perspective views of the ultrasonic probe 1 of the present embodiment. Specifically, FIG. 2 is a perspective view in which the ultrasonic probe 1 is viewed from an upper surface side. FIG. 3 is a perspective view in which the ultrasonic probe 1 is viewed from a lower surface side (upside-down direction). The lower surface (a first surface portion 11a which will be described later) of the ultrasonic probe 1 is brought into close contact with the skin surface. With reference to FIGS. 2 and 3, a configuration of the ultrasonic probe 1 will be described.

In the drawings from FIG. 2, an XYZ coordinate system is also illustrated for better understanding of the drawings. A front direction of the suction tube 35 for applying the suction pressure to the ultrasonic probe 1 is set as a +X direction (front direction), a direction which is perpendicular to the X direction, is substantially parallel to the subject (skin surface), and is a rightward direction in FIG. 2, is set to a +Y direction (rightward direction), and a direction which is perpendicular to the +X direction and the +Y direction, and is an upward direction in FIG. 2, is set as a +Z direction (upward direction). The Z direction is a thickness direction.

As illustrated in FIG. 2, the ultrasonic probe 1 of the present embodiment is configured to include an ultrasonic device 20, an accommodation case 10, and the like. The ultrasonic device 20 is constituted of an ultrasonic element, a backing material, an acoustic matching layer (none illustrated), an acoustic lens 21, and the like. The ultrasonic device 20 causes ultrasonic waves generated by the ultrasonic element to be incident to the subject via the acoustic matching layer and the acoustic lens 21. The ultrasonic device 20 receives reflected waves (ultrasonic waves) reflected from the inside of the subject, and generates a voltage corresponding to the strength of the reflected waves.

The ultrasonic element converts a transmission signal which is an electric signal into an ultrasonic wave, and converts an ultrasonic echo from a target object (subject) into an electric signal. The ultrasonic element may be, for example, a bulk piezoelectric type ultrasonic element, or may be a capacitive mircomachined ultrasonic transducer (CMUT), in addition to a thin piezoelectric type ultrasonic element of the present embodiment.

The acoustic matching layer takes acoustic matching for reducing acoustic impedance between the ultrasonic element and the subject so as to minimize reflection of ultrasonic waves and for making the ultrasonic waves to be efficiently incident to the inside of the subject. The acoustic lens 21 functions as a so-called lens which causes spread ultrasonic waves emitted from the ultrasonic element to converge so as to improve a resolution. The backing material reduces residual vibration in the ultrasonic element so as to improve a distance resolution in an image.

As illustrated in FIGS. 2 and 3, the ultrasonic device 20 is formed in a substantially rectangular plate shape. The accommodation case 10 is also formed in a substantially rectangular plate shape in the same manner as the ultrasonic device 20. The accommodation case 10 accommodates the ultrasonic device 20 in a state in which the acoustic lens 21 as a part of the ultrasonic device 20 is exposed.

The accommodation case 10 is constituted of a first accommodation member 11 and a second accommodation member 12 in the present embodiment. The first accommodation member 11 and the second accommodation member 12 are formed in a rectangular plate shape. The first accommodation member 11 and the second accommodation member 12 are disposed to overlap each other, and are joined together via an adhesive so as to be integrally formed with each other. In the accommodation case 10, both of the first accommodation member 11 and the second accommodation member 12 are formed by using synthetic resin members. However, the first accommodation member 11 and the second accommodation member 12 are not limited thereto, and may be formed by using other members (for example, a metal member).

As illustrated in FIG. 3, the first accommodation member 11 has an accommodation portion 115 which is a rectangular groove in a plan view on a first surface portion 11a, and accommodates the ultrasonic device 20 in the accommodation portion 115 and fixes the ultrasonic device 20 thereto. In a case where the ultrasonic device 20 is accommodated in the accommodation portion 115, and the acoustic lens 21 is exposed from the first surface portion 11a. In the present embodiment, a suction groove 111 is formed in four-side directions along an outer circumference of the accommodation portion 115 (outer circumference of the acoustic lens 21) on the first surface portion 11a of the first accommodation member 11. As illustrated in FIG. 2, a fixation portion 125 is formed on an end side (-X direction) of an upper surface (hereinafter, referred to as a fourth surface portion 12b) of the second accommodation member 12, and the suction tube 35 connected to the suction device 30 is fixed thereto.

FIG. 4 is a sectional view of the ultrasonic probe 1 illustrated in FIGS. 2 and 3, taken along an A direction. FIG. 5 is a sectional view of the ultrasonic probe 1 illustrated in FIGS. 2 and 3, taken along a B direction. Specifically, FIG. 4 is a schematic sectional view obtained by cutting the ultrasonic probe 1 in an X-Z plane, and FIG. 5 is a schematic sectional view obtained by cutting the ultrasonic probe 1 in a Y-Z plane.

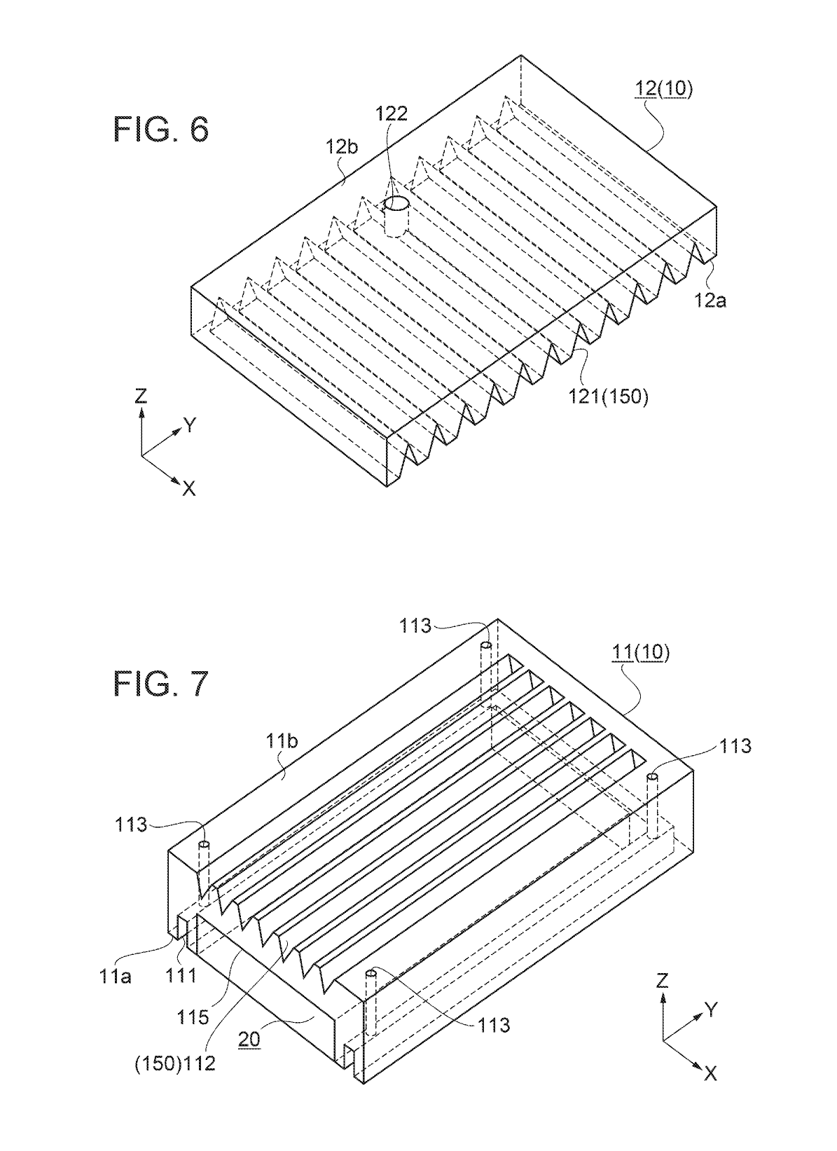

FIG. 6 is a sectional perspective view of the second accommodation member 12 in the B direction. FIG. 7 is a sectional perspective view of the first accommodation member 11 in the A direction. Specifically, FIG. 6 is a schematic perspective sectional view obtained by cutting the second accommodation member 12 in the Y-Z plane, and FIG. 7 is a schematic sectional perspective view obtained by cutting the second accommodation member 12 in the X-Z plane. In FIGS. 4 to 7, main portions which are not viewed from the outside are indicated by dashed lines.

As illustrated in FIGS. 4 and 7, the first accommodation member 11 has a plurality of first grooves 112 on a second surface portion 11b located on an opposite side to the first surface portion 11a. Each of the first grooves 112 has a section with a substantially triangular shape, and is formed to linearly extend in the Y direction as a first direction. The plurality of first grooves 112 are formed in parallel to the X direction. In the present embodiment, seven first grooves 112 are formed in the X direction as a whole. The first grooves 112 form a channel 150 along with second grooves 121 which will be described later.

The first accommodation member 11 has first communication portions 113 which communicate with the suction groove 111 and also penetrate to the second surface portion 11b. In the present embodiment, four first communication portions 113 are formed. The first communication portions 113 are respectively disposed around four corners of the second surface portion 11b. The first communication portions 113 have round hole shapes which extend upwardly (+Z direction) from the bottom 111a of the suction groove 111 and penetrate to the second surface portion 11b.

As illustrated in FIGS. 5 and 6, the second accommodation member 12 has a plurality of second grooves 121 on a third surface portion 12a facing the second surface portion 11b of the first accommodation member 11. Each of the second grooves 121 has a section with a substantially triangular shape in the same manner as the first grooves 112, and is formed to linearly extend in the X direction as a second direction. The plurality of second grooves 121 are formed in parallel to the Y direction. In the present embodiment, eleven second grooves 121 are formed in the Y direction as a whole. The second grooves 121 and the first grooves 112 are formed in directions perpendicular to each other. The second grooves 121 form the channel 150 along with the first grooves 112.

The second accommodation member 12 has a second communication portion 122 which communicates with the second grooves 121 and penetrates to the fourth surface portion 12b located on an opposite side to the third surface portion 12a. The second communication portion 122 has a round hole shape which extends upwardly (+Z direction) from an end side in the −X direction of the second groove 121 located at the nearly center among the plurality of second grooves 121, and penetrates to the fourth surface portion 12b. In FIGS. 4 to 6, the fixation portion 125 (FIG. 2) which is fixed to the fourth surface portion 12b and is provided to communicate with the second communication portion 122 is not illustrated.

The first accommodation member 11 and the second accommodation member 12 configured as described above are joined so as to overlap each other, and thus the accommodation case 10 is formed. Specifically, the second surface portion 11b of the first accommodation member 11 and the third surface portion 12a of the second accommodation member 12 are joined so as to overlap each other. Through the joining, outer circumferential regions of the second surface portion 11b and the third surface portion 12a are joined to each other without a gap. The second surface portion 11b between the first grooves 112 adjacent to each other and the third surface portion 12a between the second grooves 121 adjacent to each other are joined in a state of being intersecting to each other. As an adhesive for joining, in the present embodiment, Ablebond (trade mark of Henkel Japan Ltd.) which is an epoxy-based low-temperature curable type bond is used.

Through the joining, the plurality of first grooves 112 and second grooves 121 are in a state of being intersecting to each other in a plan view from the fourth surface portion 12b. In other words, through the joining, the first direction and the second direction are in a state of being intersecting to each other in the thickness direction of the second accommodation member 12. As illustrated in FIG. 4, the four first communication portions 113 penetrating to the second surface portion 11b of the first accommodation member 11 are located to face the second grooves 121 of the second accommodation member 12, and thus are in a state of respectively communicating with the second grooves 121.

The first grooves 112 and the second grooves 121 are joined to intersect (orthogonal to) each other, and thus a channel 150 reaching the second communication portion 122 from the first communication portions 113 is formed. In other words, the plurality of first grooves 112 which are not connected to each other and the plurality of second grooves 121 which are not connected to each other are joined to intersect (orthogonal to) each other, and thus the new channel 150 via which the respective grooves are connected to each other is formed. Consequently, a suction path is formed of the suction groove 111, the first communication portions 113, the channel 150 (the first grooves 112 and the second grooves 121), and the second communication portion 122 in the ultrasonic probe 1.

Hereinafter, an operation of the ultrasonic probe 1 of the present embodiment will be described by exemplifying a case where the ultrasonic probe 1 is used to perform puncturing (for example, catheter indwelling). A location with which the ultrasonic probe 1 is brought into close contact is the arm of a patient.

First, ultrasonic gel is applied on the surface of the acoustic lens 21 of the ultrasonic probe 1. The acoustic lens 21 is brought into contact with a skin surface of the arm of the patient at a position where the ultrasonic probe 1 is provided. After an installation position of the ultrasonic probe 1 is determined, the suction device 30 is driven. Consequently, a predetermined suction pressure generated by the suction device 30 is applied to the second communication portion 122 of the ultrasonic probe 1 via the suction tube 35.

The ultrasonic probe 1 sucks air and the gel from the suction groove 111 due to the suction pressure applied to the second communication portion 122 so as to remove an air layer generated in a gap between the ultrasonic probe 1 and the skin surface. The air and the gel sucked from the suction groove 111 flow through the channel 150 formed by the intersection between the first grooves 112 and the second grooves 121 via the first communication portions 113. At this time, the sucked gel is stored in the first grooves 112 and the second grooves 121. The sucked air flows through the channel 150, and flows from the second communication portion 122 into the suction tube 35 so as to be sucked by the suction device 30. The gel is also stored in the suction groove 111.

The space between the ultrasonic probe 1 and the skin surface can be maintained at a predetermined negative pressure by repeatedly performing this operation, and thus the ultrasonic probe 1 comes into close contact with the skin surface via the gel. Suction is continuously performed at the predetermined suction pressure generated by the suction device 30, and thus the ultrasonic probe 1 can be held at the installation position.

In this state, an operator performs a puncturing operation with both hands thereof. In a case where the puncturing operation is completed, driving of the suction device 30 is stopped. Consequently, the ultrasonic probe 1 is detached from the skin surface.

According to the above-described embodiment, the following effects can be achieved.

According to the ultrasonic probe 1 of the present embodiment, in a case where the ultrasonic probe 1 is brought into contact with a skin surface of a subject via gel, and air between the ultrasonic probe 1 and the skin surface is sucked by the ultrasonic probe 1, the gel sucked along with the air can be stored in the first grooves 112 and the second grooves 121 via the channel 150 formed by the intersection between the first grooves 112 and the second grooves 121. The air can be sucked via the channel 150. With this configuration, the ultrasonic probe 1 can be brought into close contact with a skin surface so as to be held at a desired position.

According to the ultrasonic probe 1 of the present embodiment, a plurality of first grooves 112 and second grooves 121 are formed, and thus it is possible to increase a capacity for storing gel. Consequently, the ultrasonic probe 1 can be brought into close contact with a subject so as to be held more stably.

According to the ultrasonic probe 1 of the present embodiment, a sectional shape of each of the first grooves 112 and the second grooves 121 has a triangular shape. Consequently, even if the first accommodation member 11 and the second accommodation member 12 forming the accommodation case 10 are thin, since a thick portion in the sectional direction (Z direction) can be secured, and the rigidity of the accommodation case 10 can be secured, it is possible to achieve thinning of the accommodation case 10.

According to the ultrasonic probe 1 of the present embodiment, the first grooves 112 and the second grooves 121 are formed of a plurality of linear grooves in parallel to each other, and are intersecting to each other through joining between the first accommodation member 11 and the second accommodation member 12. According to the ultrasonic probe 1, the grooves (the first grooves 112 and the second grooves 121) can be efficiently disposed, and thus it is possible to increase a capacity for storing gel and also to secure the rigidity of the accommodation case 10 through joining therebetween. With this configuration, it is possible to simultaneously achieve conflicting objects such as an increase of the storage capacity and securing of the rigidity with good balance and at a high level.

According to the ultrasonic probe 1 of the present embodiment, in a case where the ultrasonic probe 1 is brought into contact with a skin surface of a subject via gel, and air is sucked from the second communication portion 122, air in a gap between the ultrasonic probe 1 and the skin surface is sucked via the channel 150 (the second grooves 121 and the first grooves 112), the first communication portions 113, and the suction groove 111. The gel sucked along with the air is stored in the first grooves 112 and the second grooves 121 intersecting each other in a plan view, via the suction groove 111 and the first communication portions 113, and thus the air can be sucked via the second communication portion 122.

According to the ultrasonic probe 1 of the present embodiment, the suction path is formed of the second communication portion 122, the channel 150 (the second grooves 121 and the first grooves 112), the first communication portions 113, and the suction groove 111. Sucked gel is stored in the first grooves 112 and the second grooves 121, and thus air can be made to flow toward the suction device 30. With this configuration, it is possible to easily form the suction path, and thus to hold the ultrasonic probe 1 on a skin surface of a subject.

According to the ultrasonic probe 1 of the present embodiment, four (a plurality of) first communication portions 113 are formed in the present embodiment, and thus it is possible to reduce time to bring the ultrasonic probe 1 into close contact with a subject. In the present embodiment, since the first communication portions 113 are disposed around the four corners of the second surface portion 11b, and thus suction pressure (negative pressure) can be substantially uniformly applied to a skin surface, it is possible to prevent the ultrasonic probe 1 from being deviated relative to a predetermined position due to a pressure difference during suction.

According to the ultrasonic image apparatus 100 of the present embodiment, since the ultrasonic probe 1 and the suction device 30 are connected to each other via the suction tube 35, and thus suction pressure generated by the suction device 30 is applied to the second communication portion 122 of the ultrasonic probe 1, the ultrasonic probe 1 can be brought into close contact with a subject so as to be held thereon. An input signal from the ultrasonic probe 1 is processed by the processing device 40, and a generated image is displayed on the display device 41. Consequently, it is not necessary for an operator to hold the ultrasonic probe 1 with the hand, and thus it is possible to perform an optimal puncturing operation by using both hands while checking an image on the display device 41 in an appropriate attitude.

The ultrasonic probe 1 of the present embodiment is thin, small, and light weight, and is not limited in an installation location on a subject. In addition, the ultrasonic probe 1 can be brought into close contact with skin via gel so as to be held at a desired position. Therefore, it is possible to reduce influence on the subject due to the weight of the ultrasonic probe 1 or close contact, and thus to perform a puncturing operation on the subject in a state close to a natural state. For example, in a case where a vein is punctured, it is possible to prevent the occurrence of a situation in which the vein is destroyed due to a pressure caused by sticking in a case where an ultrasonic probe is stuck to a skin surface with a tape or the like as in the related art. Consequently, by using the ultrasonic probe 1 (ultrasonic image apparatus 100) of the present embodiment, puncturing can be performed while checking a position, a direction, and a depth of the vein and also to safely and reliably indwell a guide wire or a catheter into the vein while checking arrival of a puncture needle at the vein or penetration thereof.

The ultrasonic probe 1 of the present embodiment and the ultrasonic image apparatus 100 using the ultrasonic probe 1 can be suitably used for a nerve block therapy, biopsy, radiofrequency ablation (RFA), blood sampling, and carotid artery echo inspection.

### Second Embodiment

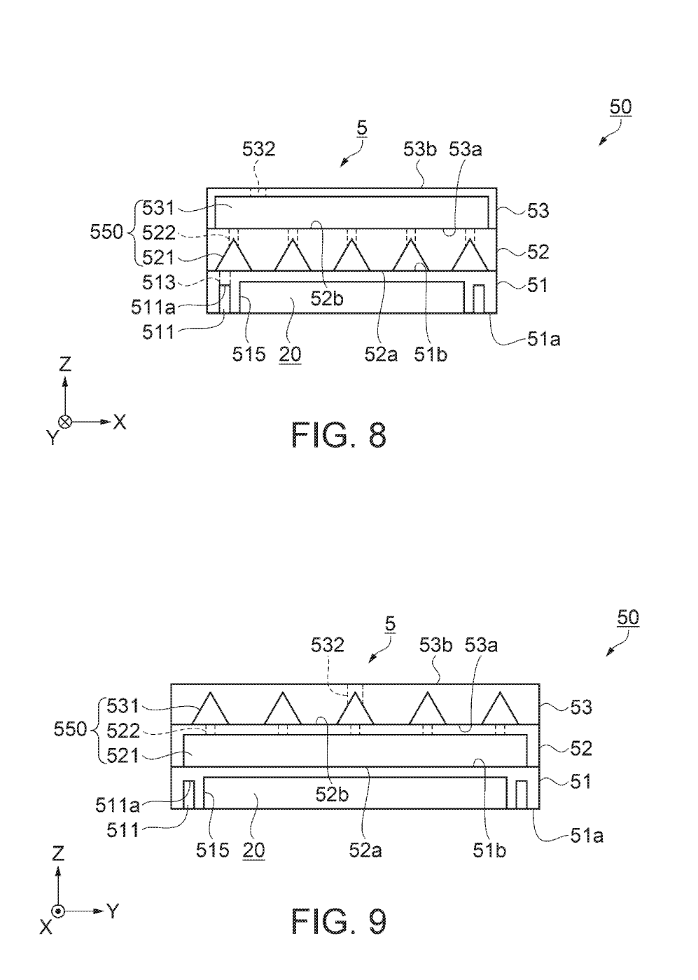

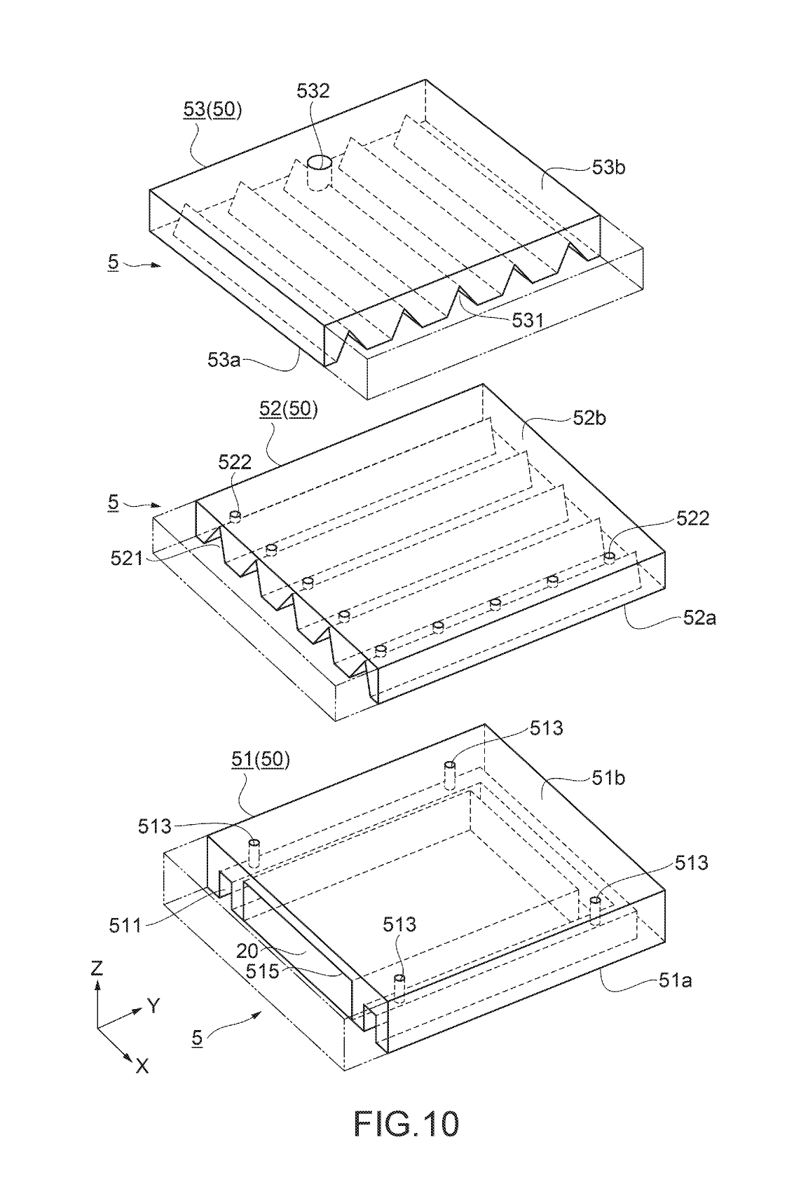

FIGS. 8 and 9 are sectional views of an ultrasonic probe 5 according to a second embodiment. Specifically, Fig. is a schematic sectional view obtained by cutting the ultrasonic probe 5 in an X-Z plane, and FIG. 9 is a schematic sectional view obtained by cutting the ultrasonic probe 5 in a Y-Z plane. FIG. 10 is a sectional perspective view of the ultrasonic probe 5. In FIG. 10, a front-stage accommodation member 51 and a first accommodation member 52 are illustrated in a schematic perspective view obtained when cut in the X-Z plane, and a second accommodation member 53 is illustrated in a schematic perspective view obtained when cut in the Y-Z plane. In FIGS. 8 to 10, main portions which are not viewed from the outside are indicated by dashed lines. A cut exterior is simply indicated by a two-dot chain line. With reference to FIGS. 8 to 10, a configuration of the ultrasonic probe 5 will be described.

The ultrasonic probe 5 of the present embodiment is different from the ultrasonic probe 1 of the first embodiment in terms of a configuration of an accommodation case 50. The ultrasonic device 20 forming the ultrasonic probe 5 is the same as that in the first embodiment. The ultrasonic probe 5 of the present embodiment is replaced with the ultrasonic probe 1 of the first embodiment, and thus the same ultrasonic image apparatus 100 as in the first embodiment is configured.

The accommodation case 50 of the present embodiment is constituted of three members such as the front-stage accommodation member 51, the first accommodation member 52, and the second accommodation member 53. Each of the front-stage accommodation member 51, the first accommodation member 52, and the second accommodation member 53 is formed in a rectangular plate shape.

The front-stage accommodation member 51 has a rectangular accommodation portion 515 which accommodates the ultrasonic device 20 on a first surface portion 51a. A suction groove 511 is formed in four-side directions along an outer circumference of the accommodation portion 515 on the first surface portion 51a. The front-stage accommodation member 51 has first communication portions 513 which communicate with the suction groove 511 and also penetrate to a second surface portion 51b located on an opposite side to the first surface portion 51a. In the present embodiment, four first communication portions 513 are formed. The first communication portions 513 have round hole shapes which extend upwardly (+Z direction) from the bottom 511a of the suction groove 511 and penetrate to the second surface portion 51b. The first communication portions 513 are provided by two so as to correspond to respective first grooves 521 located on both end sides in the X direction, which will be described later.

The first accommodation member 52 has a plurality of first grooves 521 on a third surface portion 52a facing the second surface portion 51b of the front-stage accommodation member 51. Each of the first grooves 521 has a section with a substantially triangular shape, and is formed to linearly extend in the Y direction as a first direction. The plurality of first grooves 521 are formed in parallel to the X direction. In the present embodiment, five first grooves 521 are formed in the X direction as a whole. The respective first grooves 521 located on both end sides in the X direction are disposed to face the first communication portions 513 penetrating to the second surface portion 51b of the front-stage accommodation member 51 and can communicate therewith.

The first accommodation member 52 has intermediate communication portions 522 which communicate with the first grooves 521 and also penetrate to a fourth surface portion 52b located on an opposite side to the third surface portion 52a. In the present embodiment, the intermediate communication portions 522 are formed by at least one so as to correspond to the plurality of first grooves 521 arranged in the X direction. The intermediate communication portions 522 are formed by one so as to correspond to each of a plurality of second grooves 531, which will be described later, in the Y direction in which the first grooves 521 extend, for a single first groove 521 located at the end in the +X direction.

The second accommodation member 53 has a plurality of second grooves 531 on a fifth surface portion 53a facing the fourth surface portion 52b of the first accommodation member 52. Each of the second grooves 531 has a section with a substantially triangular shape, and is formed to linearly extend in the X direction as a second direction. The plurality of second grooves 531 are formed in parallel to the Y direction. In the present embodiment, five second grooves 531 are formed in the Y direction as a whole.

The second groove 531 located on the end side of the second grooves 531 in the -Y direction is located to face the intermediate communication portions 522 which are located on the end side of the fourth surface portion 52b in the -Y direction and penetrate thereto, and can communicate with the intermediate communication portions 522. The second groove 531 located on the end side of the second grooves 531 in the +X direction is located to face the intermediate communication portions 522 which are located on the end side of the fourth surface portion 52b in the +X direction and penetrate thereto, and can communicate with the intermediate communication portions 522.

The second accommodation member 53 has a second communication portion 532 which communicates with a -X direction end side of the second groove 531 located at the center among the plurality of second grooves 531, and penetrates to a sixth surface portion 53b located at an opposite side to the fifth surface portion 53a. FIGS. 8 to 10 do not illustrate a fixation portion which is provided on the sixth surface portion 53b, communicates with the second communication portion 532, and fixes the suction tube 35 of the suction device 30 thereto.

The front-stage accommodation member 51, the first accommodation member 52, and the second accommodation member 53 configured as described above are sequentially joined so as to overlap each other, and thus the accommodation case 50 is formed. Specifically, the second surface portion 51b of the front-stage accommodation member 51 and the third surface portion 52a of the first accommodation member 52 are joined so as to overlap each other. Through the joining, the second surface portion 51b and the third surface portion 52a are joined to each other without a gap. Next, the fourth surface portion 52b of the first accommodation member 52 and the fifth surface portion 53a of the second accommodation member 53 are joined to overlap each other. Through the joining, the fourth surface portion 52b and the fifth surface portion 53a are joined to each other without a gap. As an adhesive for joining, in the present embodiment, Ablebond which is an epoxy-based low-temperature curable type bond is used in the same manner as in the first embodiment.

Through the joining, the plurality of first grooves 521 and second grooves 531 are in a state of being intersecting to each other in a plan view from the sixth surface portion 53b. In other words, through the joining, the first direction and the second direction are in a state of being intersecting to each other in the thickness direction of the second accommodation member 53. As illustrated in FIG. 8, the four first communication portions 513 penetrating to the second surface portion 51b of the front-stage accommodation member 51 are located to face the first grooves 521 of the first accommodation member 52, and thus are in a state of respectively communicating with the first grooves 521. The nine intermediate communication portions 522 penetrating to the fourth surface portion 52b of the first accommodation member 52 are located to face the second grooves 531 of the second accommodation member 53, and thus in a state of respectively communicating with the second grooves 531.

The first grooves 521 and the second grooves 531 are joined to intersect (orthogonal to) each other via the intermediate communication portions 522, and thus a channel 550 reaching the second communication portion 532 from the first communication portions 513 is formed. In other words, the plurality of first grooves 521 which are not connected to each other and the plurality of second grooves 531 which are not connected to each other are joined to intersect (orthogonal to) each other, and thus the new channel 550 via which the respective grooves communicate with the intermediate communication portions 522 and are connected to each other is formed. Consequently, a suction path is formed of the suction groove 511, the first communication portions 513, the channel 550 (the first grooves 521, the intermediate communication portions 522, and the second grooves 531), and the second communication portion 532 in the ultrasonic probe 5.

An operation of the ultrasonic probe 5 of the present embodiment will be described in the same premise as in the first embodiment.

First, ultrasonic gel is applied on the surface of the acoustic lens 21 of the ultrasonic probe 5. An installation position of the ultrasonic probe 5 is determined, the ultrasonic probe 5 is brought into contact with a skin surface of the arm of the patient, and the suction device 30 is driven. Consequently, a predetermined suction pressure generated by the suction device 30 is applied to the second communication portion 532 of the ultrasonic probe 5 via the suction tube 35.

The ultrasonic probe 5 sucks air and the gel from the suction groove 511 due to the suction pressure applied to the second communication portion 532 so as to remove an air layer generated in a gap between the ultrasonic probe 5 and the skin surface. The ultrasonic probe 5 sucks the air and the gel from the suction groove 511.

The air and the gel sucked from the suction groove 511 flow through the channel 550 (the first grooves 521, the intermediate communication portions 522, and the second grooves 531) via the first communication portions 513. At this time, the sucked gel is stored in the first grooves 521 and the second grooves 531. The sucked air flows through the channel 550, and flows from the second communication portion 532 into the suction tube 35 so as to be sucked by the suction device 30. The gel is also stored in the suction groove 511.

The space between the ultrasonic probe 5 and the skin surface can be maintained at a predetermined negative pressure by repeatedly performing this operation, and thus the ultrasonic probe 5 comes into close contact with the skin surface via the gel. Suction is continuously performed at the predetermined suction pressure generated by the suction device 30, and thus the ultrasonic probe 5 can be held at the installation position.

In this state, an operator performs a puncturing operation with both hands thereof. In a case where the puncturing operation is completed, driving of the suction device 30 is stopped. Consequently, the ultrasonic probe 5 is detached from the skin surface.

According to the ultrasonic probe 5 of the above-described embodiment, and the ultrasonic image apparatus 100 including the ultrasonic probe 5, the same effect as in the ultrasonic probe 1 of the first embodiment and the ultrasonic image apparatus 100 including the ultrasonic probe 1 can be achieved.

The invention is not limited to the above-described embodiments, and may be variously modified or altered within the scope without departing from the spirit thereof. Modification examples will be described below.

In the ultrasonic probe 1 of the first embodiment, the second communication portion 122 penetrates to the fourth surface portion 12b located on an opposite side to the third surface portion 12a. However, the invention is not limited thereto, and the second communication portion 122 may penetrate to a surface portion in contact with the third surface portion 12a. In this case, the fixation portion 125 is provided on the surface portion to which the second communication portion 122 penetrates, so as to be connected to the suction tube 35, and thus it is possible to reduce a thickness of the ultrasonic probe 1. Similarly, in the ultrasonic probe 5 of the second embodiment, the second communication portion 532 penetrates to a surface portion in contact with the fifth surface portion 53a, a fixation portion is provided on the surface portion so as to be connected to the suction tube 35, and thus it is possible to reduce a thickness of the ultrasonic probe 5.

In the ultrasonic probe 1 of the first embodiment, the number of grooves in the first grooves 112 and the second grooves 121 is not limited to the present embodiment. Similarly, in the ultrasonic probe 5 of the second embodiment, the number of grooves in the first grooves 521 and the second grooves 531 is not limited to the present embodiment.

In the ultrasonic probe 1 of the first embodiment, the first grooves 112 and the second grooves 121 are brought into a state of being intersecting to each other in a plan view as a result of the first accommodation member 11 and the second accommodation member 12 being joined to each other, but are not limited to being orthogonal and may intersect each other. Similarly, in the ultrasonic probe 5 of the second embodiment, the first grooves 521 and the second grooves 531 are brought into a state of being intersecting to each other in a plan view, but are not limited to being orthogonal and may intersect each other.

In the ultrasonic probe 1 of the first embodiment, the first communication portions 113 communicate with the suction groove 111 and penetrate to the second surface portion 11b. However, the invention is not limited thereto, and the first communication portions 113 may communicate with the suction groove 111 and may be communicate with the first grooves 112.

In the ultrasonic probe 1 of the first embodiment, the first communication portions 113 communicate with the suction groove 111, and penetrate to the second surface portion 11b. However, the invention is not limited thereto, and the suction groove 111 may have the function of the first communication portions 113 as a result of the suction groove 111 being partially extended in a depth direction (+Z direction) so as to penetrate to the second surface portion 11b or to communicate with the first grooves 112.

In the ultrasonic probe 1 of the first embodiment, a sectional shape of each of the first grooves 112 and the second grooves 121 is a triangular shape. However, the invention is not limited thereto, and a sectional shape may not be a triangular shape as long as the necessary rigidity and the capacity for storing gel can be secured by joining the first accommodation member 11 and the second accommodation member 12 to each other. Similarly, in the ultrasonic probe 5 of the second embodiment, a sectional shape of each of the first grooves 521 and the second grooves 531 may not be a triangular shape.

What is claimed is:

1. An ultrasonic probe comprising:
an ultrasonic device that performs transmission and reception of ultrasonic waves; and
an accommodation case to which a part of the ultrasonic device is exposed and in which the ultrasonic device is accommodated,
wherein the accommodation case is formed of at least a first accommodation member and a second accommodation member,
wherein each of the first accommodation member and the second accommodation member includes a suction path which is communicable with an external suction source,
wherein the first accommodation member includes, as the suction path, a first groove extending in a first direction,
wherein the second accommodation member includes, as the suction path, a second groove extending in a second direction, and
wherein the first accommodation member and the second accommodation member are disposed to overlap each other, and the first direction and the second direction intersect each other in a thickness direction of the first accommodation member or the second accommodation member.

2. The ultrasonic probe according to claim 1,
wherein a plurality of the first grooves and/or the second grooves are formed.

3. The ultrasonic probe according to claim 1,
wherein a sectional shape of the first groove and/or the second groove is a triangular shape.

4. The ultrasonic probe according to claim 1,
wherein the first accommodation member and the second accommodation member are disposed to overlap each other, and thus the first direction and the second direction are intersecting to each other in the thickness direction.

5. The ultrasonic probe according to claim 1,
wherein the first accommodation member includes
an accommodation portion that is formed on a first surface portion and in which the ultrasonic device is accommodated;
a suction groove that is formed on the first surface portion along an outer circumference of the accommodation portion;
the first groove that is formed on a second surface portion located on an opposite side to the first surface portion, and forms a channel; and
a first communication portion that communicates with the suction groove,
wherein the second accommodation member includes
the second groove that is formed on a third surface portion overlapping the second surface portion of the first accommodation member, and forms the channel by intersecting the first groove in the thickness direction; and
a second communication portion that communicates with the second groove,
wherein the first communication portion communicates with the first groove, or penetrates to the second surface portion so as to communicate with the second groove, and
wherein the second communication portion penetrates to a surface portion in contact with the third surface portion, or penetrates to a fourth surface portion located on an opposite side to the third surface portion.

6. The ultrasonic probe according to claim 5,
wherein a plurality of the first communication portions are formed.

7. An ultrasonic image apparatus comprising:
the ultrasonic probe according to claim 1;
a suction device that performs generation and control of suction pressure;
a suction tube that connects the second communication portion to the suction device, and applies the suction pressure to the second communication portion;
a processing device that controls the ultrasonic probe, and processes an input signal from the ultrasonic probe;
a cable that connects the ultrasonic probe to the processing device, and performs transmission and reception of signals; and
a display device that displays an image generated through processing in the processing device.

8. An ultrasonic image apparatus comprising:
the ultrasonic probe according to claim 2;
a suction device that performs generation and control of suction pressure;
a suction tube that connects the second communication portion to the suction device, and applies the suction pressure to the second communication portion;
a processing device that controls the ultrasonic probe, and processes an input signal from the ultrasonic probe;
a cable that connects the ultrasonic probe to the processing device, and performs transmission and reception of signals; and
a display device that displays an image generated through processing in the processing device.

9. An ultrasonic image apparatus comprising:
the ultrasonic probe according to claim 3;
a suction device that performs generation and control of suction pressure;
a suction tube that connects the second communication portion to the suction device, and applies the suction pressure to the second communication portion;
a processing device that controls the ultrasonic probe, and processes an input signal from the ultrasonic probe;
a cable that connects the ultrasonic probe to the processing device, and performs transmission and reception of signals; and
a display device that displays an image generated through processing in the processing device.

10. An ultrasonic image apparatus comprising:
the ultrasonic probe according to claim 4;
a suction device that performs generation and control of suction pressure;
a suction tube that connects the second communication portion to the suction device, and applies the suction pressure to the second communication portion;
a processing device that controls the ultrasonic probe, and processes an input signal from the ultrasonic probe;
a cable that connects the ultrasonic probe to the processing device, and performs transmission and reception of signals; and
a display device that displays an image generated through processing in the processing device.

11. An ultrasonic image apparatus comprising:
the ultrasonic probe according to claim 5;
a suction device that performs generation and control of suction pressure;
a suction tube that connects the second communication portion to the suction device, and applies the suction pressure to the second communication portion;
a processing device that controls the ultrasonic probe, and processes an input signal from the ultrasonic probe;
a cable that connects the ultrasonic probe to the processing device, and performs transmission and reception of signals; and
a display device that displays an image generated through processing in the processing device.

12. An ultrasonic image apparatus comprising:
the ultrasonic probe according to claim 6;
a suction device that performs generation and control of suction pressure;
a suction tube that connects the second communication portion to the suction device, and applies the suction pressure to the second communication portion;
a processing device that controls the ultrasonic probe, and processes an input signal from the ultrasonic probe;
a cable that connects the ultrasonic probe to the processing device, and performs transmission and reception of signals; and
a display device that displays an image generated through processing in the processing device.
